(19) United States
(12) Patent Application Publication
(10) Pub. No.: US 2016/0266084 A1
(43) Pub. Date: Sep. 15, 2016

(54) Unified Sampling and Analytical System for Monitoring Volatile Chemicals in Ground Water, Soil-Gas and Indoor Air Quality with Sample Collection For Laboratory Analysis

(71) Applicants: Scott Russell Burge, Tempe, AZ (US); David Allan Hoffman, Tempe, AZ (US)

(72) Inventors: Scott Russell Burge, Tempe, AZ (US); David Allan Hoffman, Tampe, AZ (US)

(21) Appl. No.: 14/642,463

(22) Filed: Mar. 9, 2015

(51) Int. Cl. G01N 33/18

(52) U.S. Cl. CPC G01N 33/1826

(57) ABSTRACT

An automated unified monitoring/analytical system for the sampling and analysis of volatile compounds (VOCs) in gaseous (soil-gas and atmospheric) and water samples. The unified system uses static and dynamic headspace techniques to partition VOCs from the water samples into a gaseous sample that can be introduced into a single sensor (or sensor array) for the analysis of the VOCs. The identical sensor (or sensor array) is used for the analysis of soil-gas and atmospheric samples. The monitoring system can acquire samples from a variety of sources and medias and provide analytical information on all the sources and medias using one sensor (or sensor array).

The system can be calibrated using gaseous standards, permeation tubes or aqueous standards.

### CROSS-REFERENCE TO RELATED APPLICATIONS

Reference is made to our Provisional Application No. 61970717 filed Mar. 26, 2014, entitled “Unified Sampling and Analytical System for Monitoring Volatile Organic Compounds in Groundwater, Soil-gas and Indoor Air Quality with Sample Collection For Laboratory Analysis,” and Provisional Application No. 61950568 filed on Mar. 10, 2014 entitled “Unified Sampling and Analytical System for Monitoring Volatile Organic Compounds in Groundwater, Soil-gas and Indoor Air Quality” by the present inventors.

### BACKGROUND OF THE INVENTION

### Field of Invention

Recent actions by the US Environmental Protection Agency (EPA) have placed new emphasis on the impact to indoor air quality from volatile organic compounds originating from contaminated soils and ground waters. It is well documented that volatile hydrocarbons volatilizing from the groundwater can diffuse through the vadose (unsaturated) zone to the surface. The indoor air quality of buildings located above such an aquifer may be impacted by the diffusion of volatile contaminants. Determining the movement of volatile contaminants is difficult because no unified method of sampling and analysis of groundwater, soil-gas, and indoor air quality samples currently exists. The current baseline methodology for assessing volatile organic compounds (VOCs) contamination in groundwater, soil-gas, and indoor air quality consists of manually sampling the medias then submitting the samples for laboratory analysis. The sampling methodology for aqueous samples may include pumps or bailers. The aqueous samples are analyzed using several analytical methods (i.e. EPA Method 8260B). The sampling methodologies for atmospheres (soil-gas or indoor air quality) include using sampling pumps, sorptive tubes, or evacuated cylinders. The samples are analyzed using a variety of analytical methods (i.e. EPA Method TO-15). Differences in sampling techniques and analytical methods often make correlations between the groundwater samples and soil-gas samples difficult to determine.

A second major problem with current baseline techniques (and models for indoor air quality) is the assumption that a steady state exists for VOCs diffusing from the groundwater plume through the vadose zone and into the interiors of buildings. This assumption does not take into account barometric pumping and other factors resulting in non-steady state conditions. Most models assume steady-state conditions. A recent study indicated that sampling over longer time intervals are required to determine impacts to indoor air quality. A continuous monitoring system using a unified sampling and analytical methodology may be more effective than current baseline methods in reducing these determinate errors.

A major application for the disclosed invention is for monitoring of fracking operations. Fracking has become a commonly applied method for petroleum extraction in North America. Impacts from fracking may include water quality degradation and increased methane exposure to surrounding residences and municipalities. An automated system for determining methane concentrations in ground water and soil gas before and after the implementation of fracking at a site could determine the risks of operations. The assessment of methane from a fracking operation requires the measurement of methane in the environment over several months. An automated system will provide a low-cost method for determining the impacts of fracking.

The primary disclosed system uses one sensor (or one sensor array) located at a central location to measure the concentrations of volatile hydrocarbons in atmospheres. The sampling system is capable of collecting groundwater (aqueous), soil-gas, or indoor air quality (atmospheric) samples. The sampling/analytical system uses a static or dynamic headspace technique to partition volatile chemicals from the aqueous samples into an atmosphere (headspace) created above the aqueous sample. Therefore, all samples are presented to the sensor (or sensor array) as volatile chemicals in atmospheres.

Additionally, the sampling/analytical system has the ability of collecting samples for laboratory analysis. This allows the sampling/analytical system to measure trends and when the highest concentrations are encountered (or other user-selected criteria), the system collects a duplicate sample for laboratory analysis.

The disclosed sampling/analytical system calibrates the sensor (or array of sensors) using either aqueous standards, or gas standards. The gas standards are generated using standard gases (i.e. mixtures in cylinders) or gas permeation tubes. Aqueous standards are used to create an equilibrium concentration of the analyte (or analytes) in the headspace created above aqueous solutions.

The system has the capability of collecting samples or duplicates using summa canisters for laboratory analysis. The collection of laboratory samples is used for two primary purposes: 1) method of data validation, and 2) method for determining the individual VOCs present in the air samples. The collection of laboratory samples may aid in regulatory acceptance of the invention.

### BACKGROUND-PRIOR ART

The following is a tabulation of relevant prior art:

U.S. Patents

| Patent No. | Kind Code | Issue Date | Patentee |
|---|---|---|---|
| 5,646,863 | A | July 1997 | Morton |
| 6,021,664 | | Feb. 8, 2000 | Granato et al. |
| 6,936,156 | B2 | Aug. 30, 2005 | Smith et al. |
| 7,247,278 | B2 | Jul. 24, 2007 | Burge et al. |

### 2. DISCUSSION OF PRIOR ART

The field of automated monitoring systems is mature with a history of prior art spanning over 30 years. Many commercialized versions are readily available on the current market. The advent of automated instrumentation was made possible by the availability of affordable microprocessors in the early 1980s. The prior art describing monitoring methods supporting multiple sensors includes:

U.S. Pat. No. 7,247,278 describes a monitoring system to transfer groundwater samples from a well to an analytical sensor located at the surface, and methods for calibrating the sensor at the surface. Multiple analytical chambers, multiple calibration systems or deployment of alternative sensors were not disclosed.

U.S. Pat. No. 5,646,863 describes a monitoring system with a series of flow-through measuring cells for measuring multiple analytes. The invention does not describe interchangeable chambers, interchangeable calibration modules, or the measurement of the sample volumes delivered to the chambers. The sampling system continuously flows the aqueous sample through the sample chamber for analysis by the analytical sensors. The flow-through design limits the types of analytical methods that may be performed by the system. The system does not allow for the expansion of the system for additional future sensors. The system does not disclose the ability to sample and analyze multiple medias: atmospheric and aqueous samples.

U.S. Pat. No. 6,021,664 describes a flow-through system that has one sample cell with several sensors (temperature, conductance, dissolved oxygen, pH, and ammonium). No reference is made for measuring the volume within the sample cell, or to an interchangeable cell for the measurement of other contaminants. Most of the disclosure is associated with purging groundwater wells. The sampling system flows the sample through the sample chamber for analysis by the analytical sensors. The system does not disclose the ability to sample and analyze multiple medias: atmospheric and aqueous.

U.S. Pat. No. 6,936,156 describes a flow-through system with the capability of recirculating the sample through the sample cells. The system does not describe multiple sample chambers each capable of measuring the volumes delivered to the chamber. Additionally, the invention does not describe a method for incorporating additional sample chambers or cells, or the calibration of the sensors in the additional cells. The sampling system flows the sample through the sample chamber, or re-circulates the sample for analysis through the sample chamber. The system does not disclose the ability to sample and analyze multiple medias: atmospheric and aqueous.

Most of the commercial instruments (Hach) describe flow-through cells with the ability to calibrate the system by the injection of standards into the flow-through systems. The systems do not have the ability to sample and analyze multiple medias: atmospheric and aqueous.

### BRIEF DESCRIPTION OF INVENTION

The invention described in this disclosure is a monitoring system capable of collecting and analyzing volatile chemicals in atmospheric and aqueous samples from multiple sources. The aqueous sources include surface, ground, and industrial water samples. Samples are collected from atmospheres (air sample collected above the surface), and soil-gas (air collected from below the surface) using pumps (diaphragm, turbine and other devices capable of transferring gaseous samples from the source to the analytical sensor).

The volatile chemicals in water samples (i.e. groundwater) are partitioned from the aqueous sample into a headspace using either static or dynamic methods. Static methods require the aqueous sample (with dissolved volatile chemicals) to be gently agitated (i.e. stirring) at constant temperature, pressure and volume allowing portioning into a headspace (constant volume of air) situated above the aqueous sample. The partitioning continues until equilibrium is attained between the volatile chemicals in the aqueous phase and the gaseous phase. At equilibrium, the volatile chemicals concentrations between the two phases will remain constant and a measurement of the volatile chemicals equilibrium concentration in the headspace can be correlated with the original volatile concentration in the aqueous sample.

Dynamic partitioning uses a recirculating stream of a gas (i.e. air, nitrogen) passing though the sample. This process is often referred to as sparging. The recirculation of the gas through the sample will result in the establishment of an equilibrium concentration for the volatile chemicals between the aqueous and gaseous phases.

The partitioning of volatile chemicals from the aqueous phase into a gaseous (headspace) phase results in an equilibrium concentration capable of being introduced to an analytical sensor (or sensor array). The sensor (or sensor array) is capable of measuring the volatile chemicals in both atmospheric samples and aqueous samples (after partitioning into the headspace). The combination of three types of gaseous samples (atmospheric, soil-gas, and partitioned ground water samples) can be introduced to a single sensor (or sensor array). A sampling system using one sensor (or sensor array) substantially decreases the determinant error associated with investigations using multiple types of sampling devices and analytical methods for each media.

An important aspect of any analytical protocol is the ability to interrogate the sensors at frequent intervals using standards. The interrogation may be accomplished by using multiple concentrations of a standard to create a calibration curve, or by using one standard and a blank to calculate a calibration factor.

The calibration of the system can be accomplished using the introduction of a blank and a spiked blank. A blank is defined as a gaseous standard (devoid of volatile chemicals) periodically introduced into the sensor (or sensor array) for the purpose of the measurement of the signal with no target analytes present. A blank may be generated by: 1) passing air (or other gas) through a filter (i.e. activated carbon) to remove the volatile chemicals, 2) a blank (purchased from a commercial source), or 3) source where the volatile chemicals are known to be absent (or below the detection limits of the sensor) such as atmospheric air.

A spiked blank or standard gas is used to determine the response (signal) of the sensor to a known concentration of the target volatile chemical. A standard gas is usually purchased from a commercial source with a known concentration of a volatile chemical in air or nitrogen. A spiked blank is defined to include the introduction of a known concentration of a volatile chemical into a blank gas. One embodiment of a spiked blank is the creation of a spiked blank using a gas permeation tube. Gas permeation tubes are commercially available polymer capsules filled with the target analyte (such as trichloroethene) capable of generating a known rate (mass of the analyte per unit of time) at a given temperature (typically 30° C.). A typical rate is in micrograms/minute. A blank gas (at a constant and known rate) is passed through a heated chamber containing the permeation tube to create the spiked blank. The sensor system array may be calibrated by the measurement of the blank and spiked blank (or standard gas) and calculation of a response factor. The use of multiple calibration gases and/or permeation tube chambers will allow the generation of a calibration curve.

### SUMMARY AND ADVANTAGES

The primary advantage of the invention is the decrease in determinant error during investigations of volatile chemicals present in multiple medias.

### BRIEF DESCRIPTION OF DRAWINGS

FIG. 1 illustrates the overall monitoring/analytical system with the ability to collect atmospheric, soil-gas and water samples and transfer the samples into a sample chamber contained a single sensor (or sensor array). The sample chamber has the ability to partition volatile chemicals from the water sample into the headspace using a static headspace method.

FIG. 2 illustrates the overall monitoring/analytical system with the ability to collect atmospheric, soil-gas and water samples and transfer the samples into a chamber containing a single sensor (or sensor array). The sample chamber has the ability to partition volatile chemicals from a water sample into the headspace using a dynamic headspace method.

FIG. 3 illustrates a monitoring system with the capability of introducing liquid standards into the sample chamber.

FIG. 4 illustrates a monitoring system with the capability of introducing gaseous standards generated from a permeation tube.

FIG. 5 illustrates a monitoring system configured with both static and dynamic headspace techniques to collect duplicate samples using evacuated cylinders.

FIG. 6 illustrates a monitoring system configured with gaseous sampling techniques to collect duplicate samples using evacuated cylinders.

### DETAILED DESCRIPTION

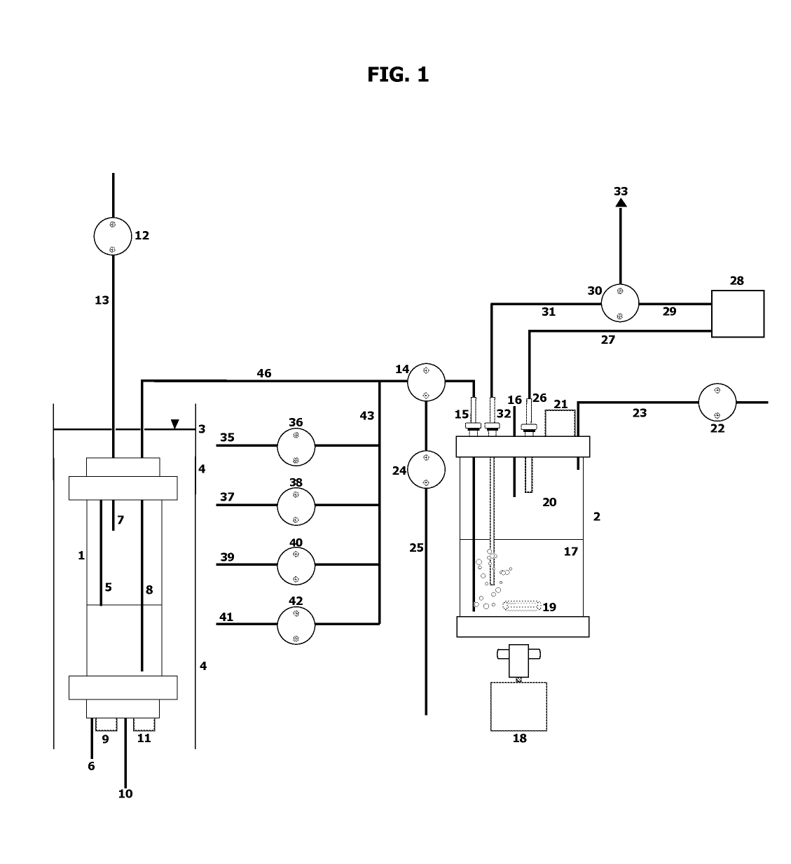

### Referring to FIG. 1

This embodiment is for the design of a two-chamber system for sampling and analysis of volatile chemicals in aqueous and gaseous (soil-gas and indoor air quality) samples. The basic design allows for the transfer of a water sample from a sampling chamber 1 to an analytical chamber 2. The typical deployment of the sampling chamber 1 is to be located 2 to 3 feet below a static water level 3 of a monitoring well 4. The sampling chamber 1 has three primary ports and a water level sensor 5. The ports include: 1) a sample inlet port 6, 2) an air pressure/vent port 7, a sample outlet port 8. The sample inlet port 6 allows water from the well 4 to be introduced into the interior of a chamber 1 using hydrostatic pressure, because the sampling chamber 1 is located below the static water level 3. The activation of an inlet port valve 9 allows water to flow into the interior of the sample chamber 1. The water flows into the chamber 1 until the water level sensor 5 located within the chamber 1 is satisfied. This action terminates the filling of the chamber 1 by closing the valve 9. This design allows for the precise volume of water to be contained within the sample chamber 1. The design allows for multiple sample inlet ports 6, 10 and sample inlet valves 9, 11.

The water sample in the chamber 1 is transferred to the analytical chamber 2 by the activation of a pressure/vent valve 12. The valve 12 connects to the air pressure/vent port 7 with a pressure/vent tube 13. The activation of the valve 12 pressurizes the headspace of the chamber 1 causing the water sample to be displaced through the sample outlet port 8. The sample outlet tube port 8 connects to the normally-closed port of a chamber valve 14. The common-port of the chamber valve 14 connects with a sample inlet port 15 of the analytical chamber 2.

A water level sensor 16 is located within the interior of the chamber 2. A volume of water 17 is transferred into the chamber 2 until the water level sensor 16 is satisfied. The water 17 contained within the chamber 2 is agitated with a magnetic stirrer 18 and a magnetic stir bar 19. The action allows the partitioning of volatile organic hydrocarbons into the headspace 20. A sensor 21 located within the chamber 2 measures the volatile chemicals in the headspace 20.

Alternatively, the volatiles are partitioned from the water 17 contained within the analytical chamber 2 into the headspace 20 using a sparging methodology. The sparging methodology consists of a chamber air outlet port 26 connecting to an inlet of a pump 28 with a tube 27. An outlet of the pump 28 connects to the common port of a recirculation valve 30 with a tube 29. The normally-open port of the valve 30 connects to a tube 31. The tube 31 passes through an air inlet port 32 into the interior of the sample chamber 2. The terminal end of the tube 31 extends into the volume of water 17. The sparging method requires the activation of the pump 28. The activation of the pump causes air, or other gas, to flow through the port 26 through the pump 28 and the tube 31. The air flows through the terminal end of the sparging tube 31 and bubbles (sparges) through the volume of water 17 contained with the chamber 2. The sparging action causes the volatile chemicals to partition from the water into the headspace 20.

The volume of water 17 in the chamber 2 is purged from the chamber 2 to waste using the following method. The chamber valve 14 connects to a waste valve 24. The waste valve 24 connects to the waste tube 25. A purge valve 22 connects to a tube 23 that passes through the wall of the chamber 2 allowing the introduction of air pressure. The volume of water 17 in the chamber 2 is purged by the activation of the valve 22 causing an increase of air pressure in the headspace 20 of the analytical chamber 2. The valves 14 and 24 are activated allowing a path for the water under pressure to be conducted from the chamber 2 through a waste tube 25 to waste. The procedure is continued until the volume of water 17 is evacuated from the sample chamber 2.

Referring to FIG. 1 the indoor air or soil-gas sampling is performed with the following components: An air tube 35 connects to the inlet of an air-sampling valve 36. Multiple air sampling valves 38, 40, 42 connects to inlet tubes 37, 39, 41, respectively. The inlet of the tube 35 connects to a source of soil-gas or indoor air. The outlet of the sampling valve 36 connects to a tube 43. The tube 43 connects to the inlet of the valve 14. The outlet of the valve 14 connects to the interior of the sample chamber 2 through the port 15. The interior of the chamber 2 connects to the chamber outlet port 26. The chamber outlet port 26 connects to the inlet of the pump 28 with the tube 27. The outlet of the pump 28 connects to the common port of the valve 30 with the tube 29. The normally-closed port of the valve 30 connects to the vent tube 33.

The operation of the soil-gas or indoor air sampling system consists of activating the sample valve 36, the vent valve 30 and the pump 28. This action allows the flow of sample air to pass through the valve 36, the tube 43, the port 15, and into the sample chamber 2. The sensor 21 detects the volatile chemicals in the air stream. The air in the chamber 2 is vented through the port 26, the tube 27 into the inlet of the pump 28. The air is discharged from the outlet of the pump 28 through the tube 29 and through the activated valve 30. The air passes through the vent tube 33 to the atmosphere.

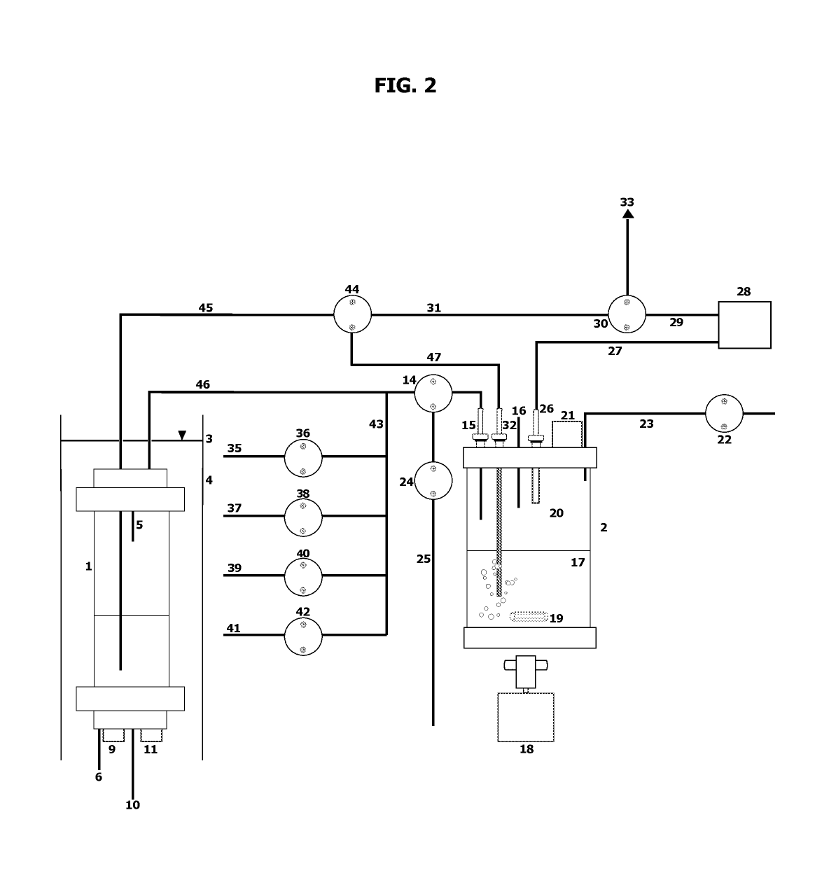

### Referring to FIG. 2

The FIG. 2 presents a system where the water sample is sparged in the sample chamber 1 located within the monitoring well 4. The typical field deployment of the sample chamber 1 is located 2 to 3 feet below the static water level 3 within the monitoring well 4. The sample chamber 1 has three primary ports and the water level sensor 5. The ports include 1) the sample inlet 6, 2) a recirculation tube 45, and 3) the tube 46. The sample inlet port 6 allows water from the well 4 to be introduced into the sample chamber 1 using hydrostatic pressure. The activation of the sample inlet port valve 9 allows water to flow into the sample chamber 1. The water flows into the sample chamber 1 until the water level sensor 5 located within the sample chamber 1 is satisfied. This action terminates the filling of the sample chamber 1 by closing the valve 9. The design allows for the precise measurement of a volume of water sample to be contained within the sample chamber 1. The design allows for multiple sample inlet ports 6, 10 and the sample inlet valves 9, 11.

The headspace of the sample chamber 1 connects to the tube 46. The tube 46 connects to the normally-closed port of the valve 14. The common-port of the valve 14 connects into the interior of the cha 2 with the port 15. The outlet port 26 vents the interior of the chamber 2. The tube 27 connects to the inlet of the pump 28. The outlet of the pump 28 connects to the common port of the valve 30 with the tube 29. The outlet of the valve 30 connects through a tube 31 to the common port of the valve 44. The normally-open port of valve 44 connects to the recirculation tube 45. The tube 45 passes through the top of the sample chamber 1. The tube 45 terminates at the bottom of sample chamber 1.

The operation of the sparging system for the sample chamber 1 includes the activation of the pump 28. The air pressure generated by the pump 28 is conducted through the tube 29, the valve 30, the tube 31 and the valve 44. The air pressure passes through the tube 45 and the air is introduced into a water sample located within the sample chamber 1. The air bubbles through the water sample in the sample chamber 1 partitioning the volatile chemicals from the water sample into the headspace of the sample chamber 1. The air is conducted from the chamber 1 through the tube 46. The entrance of the tube 46 is located the top of the chamber 1. The air then passes through the valve 14, the port 15 and into the analytical chamber 1. The sensor 21 located within the interior of chamber 2 detects the volatile organic compounds passing through the chamber 2. The air passes through the port 26 and the tube 27 into the inlet of the pump 28. The air passes through the pump 28 and repeats the cycle to sparge the water sample located within the chamber 1. The recirculation of the air continues until the sensor 21 attains a constant signal. A constant signal indicates that an equilibrium was attained between the sample and sparge air

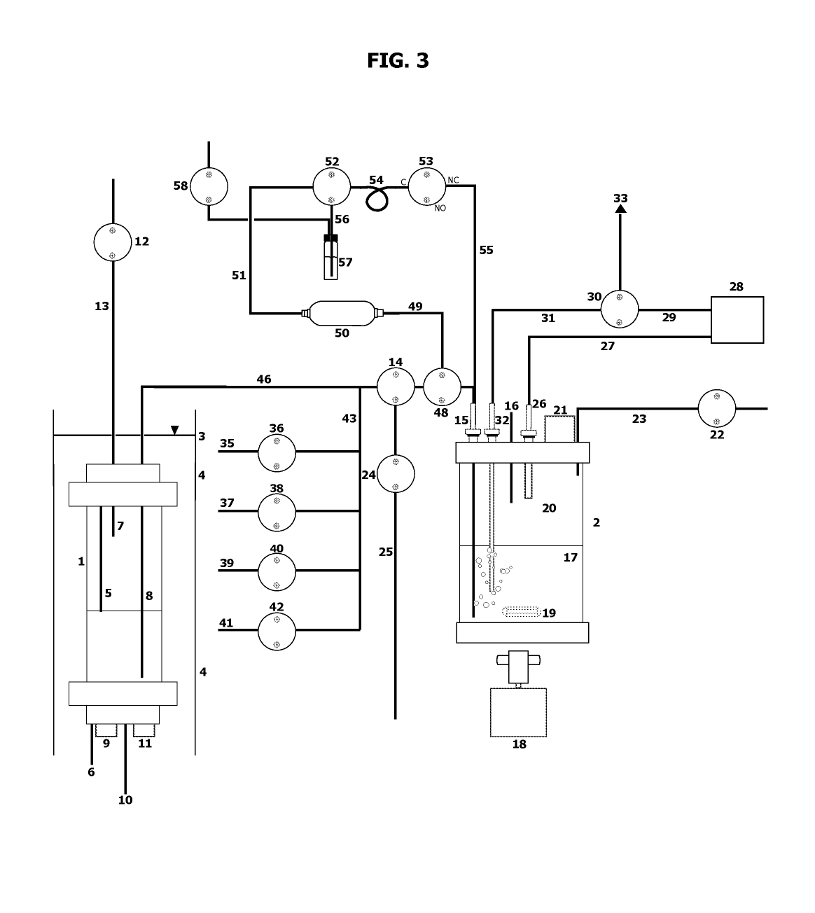

### Referring to FIG. 3

The calibration of the unified system requires the introduction of standards into the sample chamber 2. The analytical sensor 21 is located within the headspace of the sample chamber 2. The standard of the analyte of interest must be present in the headspace. The unified system was designed to use gaseous standards, permeation tubes or aqueous standards. A gaseous standard may include gas cylinders with an analyte of interest diluted in nitrogen or air. The calibration of the sensor 21 located within the chamber 2 can be accomplished in several ways.

### Gaseous Standards

The use of gaseous and permeation tube standards allows for the direct introduction of a standard of the volatile chemical into the sample chamber 2 for measurement by the analytical sensor 21.

### Aqueous Standards

The introduction of aqueous standards containing volatile chemicals requires the partitioning of the standard from the water sample 17 into the headspace within the sample chamber 2. The partitioning of volatile chemicals from the water sample 17 into the headspace is time dependent and may require several minutes to an hour to reach equilibrium. It is important that the analytical sensor 21 located within the chamber 2 is a non-destructive sensor and insensitive to water vapor. The photo-ionization detector is an acceptable sensor. The signal of the sensor is monitored during the standardization procedure to determine when an equilibrium concentration of the analyte of interest is established between the aqueous solution and the overlying headspace. A constant signal versus time for the agitated standard in the sample chamber 2 determines when equilibrium is attained. Two methods exist to establish when equilibrium of the volatile chemicals is attained. The methods include: 1) agitation of the water by stirring, and 2) sparging of the water by recirculating the headspace 20 through the analytical chamber 2. The chamber 2 is maintained at a constant temperature and pressure during the standardization procedure.

One method of calibration using aqueous standards is the introduction of groundwater into chamber 2. The same groundwater for samples must be used for calibration because the partitioning of the volatile chemicals from the water into the headspace in dependent on several factors including pressure, temperature and the concentration of dissolved ions in the solution. If the calibration is performed using the groundwater, differences in the concentration of the dissolved ions in samples and standards can be eliminated. This requires the elimination of the volatile chemicals from the water sample to be used in the calibration of the sensor. The elimination of the volatile chemicals creates the blank water. The blank water is used for direct analysis and dilution of the aqueous samples introduced into the chamber 2.

The design has two methods of removing the volatile chemicals from the sample water to create a blank: 1) passage of the water sample through an activated carbon filter, and 2) sparging of the volatile chemicals from the water in the chamber 2.

The calibration system consists of two valves and a calibrated loop to introduce a volume of aqueous standard containing volatile chemicals into the chamber 2.

Referring to FIG. 3, calibration is performed with the introduction of aqueous standards into the analytical chamber 2. The introduction of the aqueous standards requires two operations: 1) creation of a blank, and 2) introduction of a standard (volatile chemicals) into the blank.

The system for the creation of a blank includes the outlet of the valve 14 connected to a port of a calibration selection valve 48. The normally-closed port of the valve 48 connects with a tube 49 to a cartridge 50 filled with a media selected to removed volatile chemicals (i.e. activated carbon) from water. The outlet of the cartridge 50 connects to the normally-open port of a standard valve 52 with a tube 51. The common port of the valve 52 connects through a standard loop 54 to the common port of a standard waste valve 53. The normally-open port of the valve 53 connects to waste. The normally-closed port of the valve 53 connects to a tube 55 through the inlet port 15 of the analytical chamber 2.

The standard is injected into the blank using a standard bottle 57 connected to the normally-closed port of the valve 52 with a tube 56. A pressure valve 58 connects to the headspace of the standard bottle 57.

The operation of the aqueous calibration system includes a two-step process: 1) preparation of the blank, and 2) injection of the standard. The preparation of the blank water sample includes water under pressure passing through the activated valve 14, and the activated valve 50. The water is directed through the cartridge 50. The volatile chemicals are removed from the water after passing through the cartridge 50. The water passes through the unactivated valve 52, the standard loop and the unactivated valve 53. The water then passes into the analytical chamber 2. This results in the partial filling of the analytical chamber 2 with a blank.

The second step of the process, injection of standard, activates three valves including the valve 58, the valve 52 and the valve 53. The activation of the valve 58 causes air pressure to be delivered into the headspace of the standard bottle 57. The standard flows from the standard bottle 57 under pressure through the tube 56 and into the port of the activated valve 52. The standard then passes through the valve 52 through the standard loop 54 and the activated valve 53 to waste. This action sweeps the water from the standard loop 54 and creates a known volume of standard in the standard loop 54. After several seconds, the program reverts to the original valve settings used to create the blank, the first step of the process. This action sweeps the standard from the standard loop 54 into the analytical chamber 2. The program continues to fill the analytical chamber 2 with blank water until the water level sensor 16 located within the analytical chamber 2 is satisfied. This action terminates the filling of the analytical chamber 2. The result of this action introduces predetermined volumes of standard and blank water into the analytical chamber 2. The program activates the magnetic stirrer 18 until the volatile chemicals in the water 17 partitions into the headspace 20 located within the analytical chamber 2.

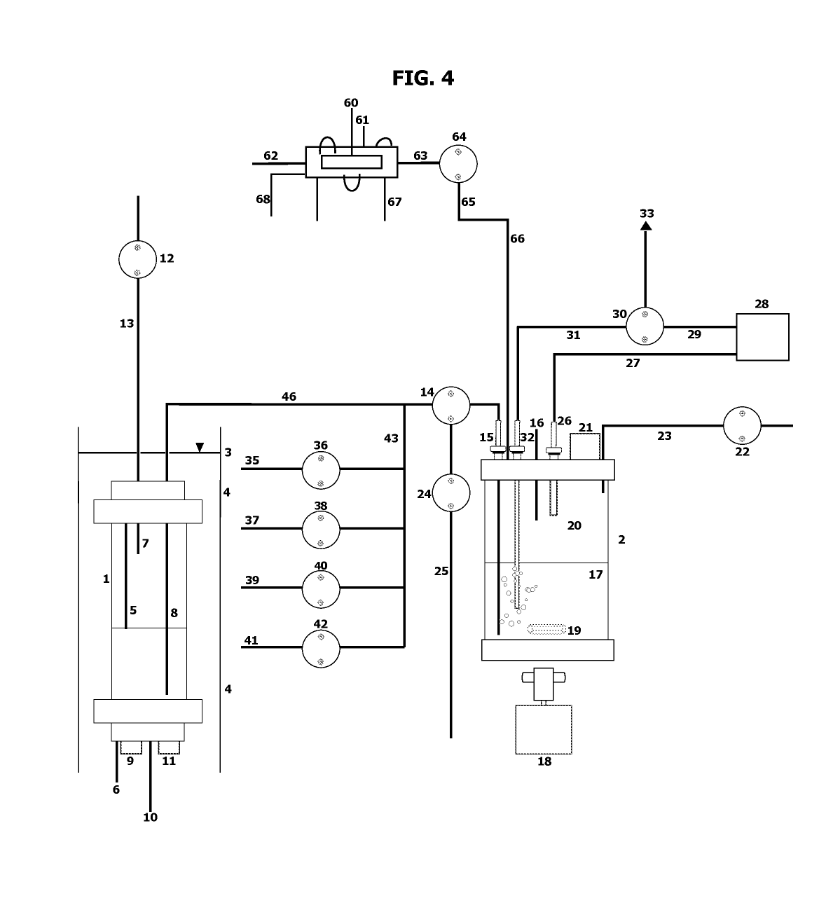

### Referring to FIG. 4

The calibration of the system with a gas permeation tube is illustrated on FIG. 4. A gas permeation tube 60 is contained within a temperature-controlled permeation tube chamber 61. A source of flow gas (air or nitrogen) passes through the chamber 61 to the common port of a permeation tube valve 64. The normally-open port of the valve 64 connects to the atmosphere. The normally-closed port of the valve 64 connects by a tube 56 to a gas permeation port 66 located on the top of the analytical chamber 2. A heater 67 and a temperature sensor 68 are attached to the exterior wall of the chamber 61.

The operation of the permeation tube calibration system includes the venting of the chamber 61 and introduction of the standard. The venting operation consists of gas flowing through the chamber 61 and the flow gas/volatile chemical mixture vents to the atmosphere through the normally-open port of the valve 64. The standard introduction consists of activating the valve 64 that diverts the flow of the gas/volatile chemical mixture through the tube 65, the port 66, and into the analytical chamber 2. The sensor 21 located within the analytical chamber 2 analyzes the flow gas/volatile chemical mixture.

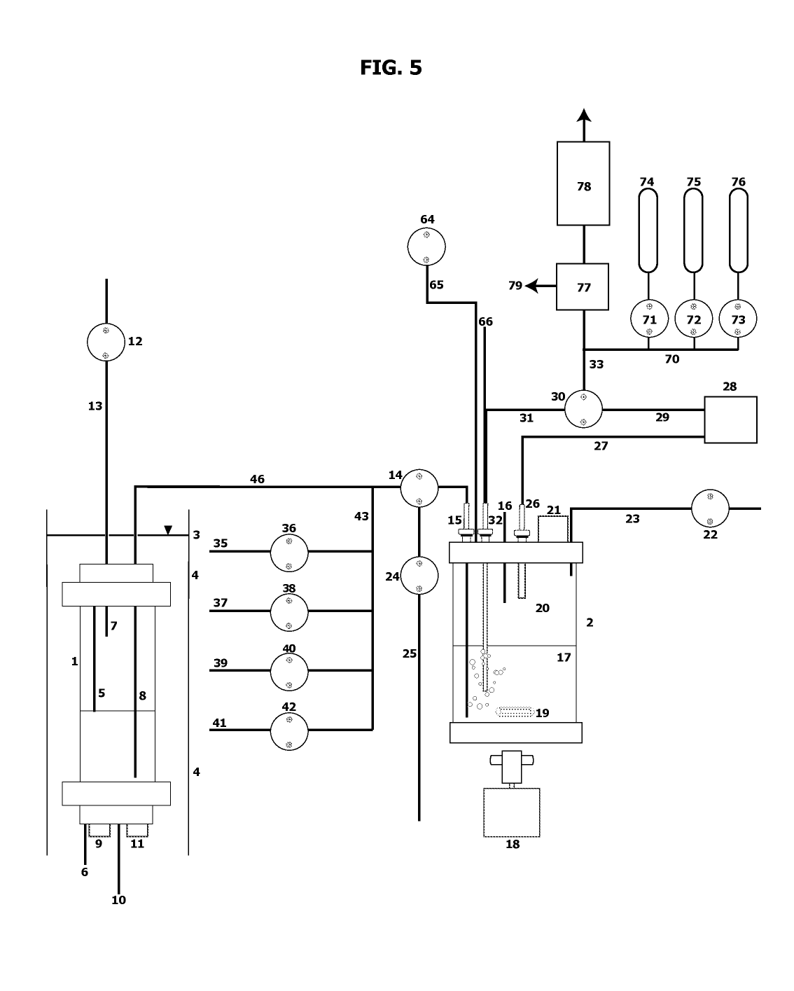

### Referring to FIG. 5

The embodiment allows for the collection of air samples using a series of valves for filling evacuated air cylinders. The primary purpose of this embodiment is to allow for better regulatory acceptance of the monitoring system, and to collect air samples with volatile chemicals below the detection limit of the sensor 21. This embodiment of the monitoring system is used primarily for: 1) trend analysis of the VOCs over time, and 2) when a predetermined user threshold is exceeded (VOC concentration measured by the analytical sensor is higher than a threshold value), the system activates a sample valve 71, 72, 73 and a sample is collected by an evacuated cylinder 74, 75, 76 for laboratory analysis.

### Referring to FIG. 5

The preferred system uses a second sensor such as an electron capture detector (ECD) for determining volatile chlorinated hydrocarbons in the air stream.

A second sensor 78 is located at the effluent of an air-stream pretreatment module 77. The air-stream pretreatment module 77 is used to remove moisture and oxygen prior to the introduction into the second detector 78. The pretreatment unit may use a Tenax column to trap volatile chemicals, and then remove the moisture and oxygen by desorption using nitrogen or another inert gas. Alternatively, the pretreatment system may use sorptive medias to remove the oxygen and moisture. The second sensor 78 is used to determine trends in the VOC concentrations in the air stream. Recent research (Johnson et al.) indicated great temporal variability in the VOC concentrations in samples collected from soil gas and indoor air. The second sensor 78 is used to determine when the VOC concentrations are present in the sample air stream to collect a laboratory sample using summa canisters.

Inlets of the sample valves 71, 72, 73 are connected to an inlet tube 70. The tube 70 connects to the tube 33 located at the outlet of the valve 30. More than three laboratory sample valves can be connected to the tube 70. The evacuated cylinders 74, 75, 76 connect to the outlet of the sample valves 71, 72, 73.

The operation of the collection of laboratory samples is performed when the sampling/analytical system acts on a user command to collect a laboratory sample. The sampling pump 28 passes air through the tube 29, the valve 30, the tube 33, and into the tube 70. The controller of the sampling/analytical system activates one of the sample valves 71, 72, 73. The action causes the air sample to pass through the activated valve 71, 72, 73 and into an evacuated cylinder 74, 75, 76.

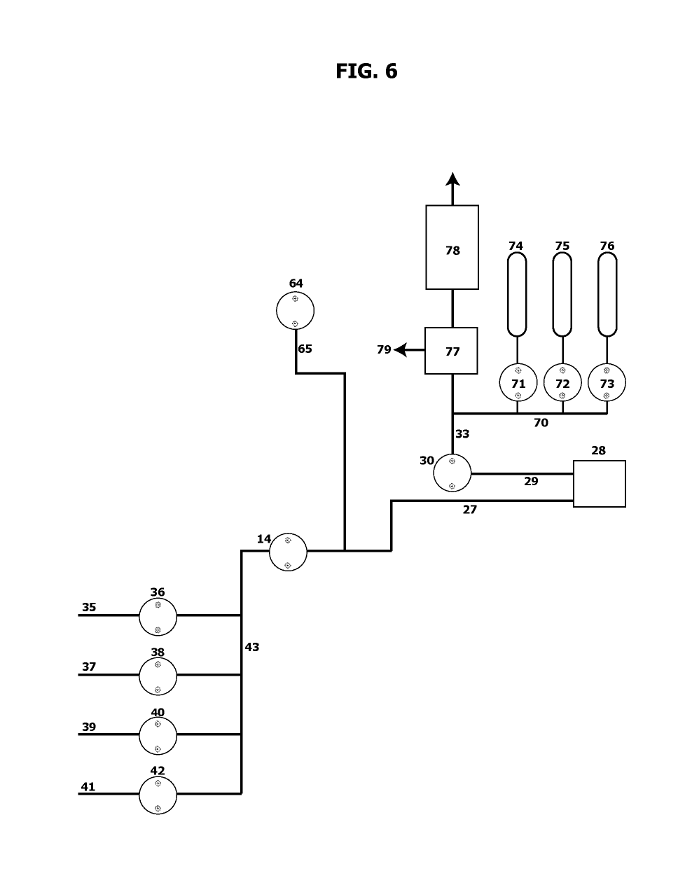

### Referring to FIG. 6

The embodiment allows for the use of a second sensor assembly and collection of air samples using a series of valves for filling evacuated air cylinders. This embodiment does not include any the components used for sampling and analysis of groundwater.

The analytical system is used primarily for trend analysis of the VOCs over time and when a predetermined user threshold is exceeded (VOC concentration measured by the analytical sensor is higher than a threshold value), the system activates one of the valves and a sample is collected for laboratory analysis.

The preferred system uses a second sensor 78 such as an electron capture detector (ECD) for determining chlorinated volatile organic hydrocarbons in the air stream.

The air samples are collected with the activation of one of the valves 36, 38, 40, 42 and the air pump 28. The air sample is conducted from the activated valve, through the tube 43, the valve 14, the tube 27 and into the pump 28. The air is pumped through the tube 29 and the valve 30. The air is conducted through tube 43 and into the entrance of the module 77.

The sensor 78 is located at the outlet of the air stream pretreatment module 67. The air stream pretreatment module 67 is used to remove moisture and oxygen prior to the introduction into the detector 68. The pretreatment unit may use a Tenax column to trap the VOCs, and then remove the moisture and oxygen by desorption using nitrogen, or another inert gas. Alternatively, the pretreatment system may use sorptive medias to remove the oxygen and moisture. The sensor is used to determine trends in the VOC concentrations in the air stream. The sensor is used to determine when the VOC concentrations are present in the sampled air stream to collect a laboratory sample using summa canisters.

Inlets of the sample valves 71, 72, 73 connect to the tube 70. The tube 70 connects to the tube 43 located at the effluent of the valve 30. There can be more that three laboratory sample valves connected to the tube 70. The evacuated cylinders 74, 75, 76 connect to the outlet of the sample valves 71, 72, 73.

The operation of the collection of laboratory samples is performed when the sampling/analytical system acts on a user command to collect a laboratory sample. The sampling pump 28 passes air past the entrance of the tube 70. The controller of the sampling/analytical system activates one of the sample valves 71, 72, 73. This action causes the air sample to pass through the activated valve 71, 72, 73 and into the evacuated cylinder 74, 75, 76.

We claim:

1. A volatile chemical monitoring system, comprising:
(a) an analytical chamber located at or near the ground surface,
(b) a sampling chamber located within a well,
(c) a means of delivering water into the sampling chamber,
(d) a means of connecting the headspace of the two chambers,
(e) a means of recirculating the headspace between the chambers,

2. said volatile chemical monitoring system of claim 1 wherein one or more sensors are located within the analytical chamber.

3. said volatile chemical monitoring system of claim 1 wherein an air pump recirculates the said headspace between the said chambers.

4. said volatile chemical monitoring system of claim 1 wherein electrical or optical sensors measure a volume of water contained within the said chambers.

5. said volatile chemical monitoring system of claim 1 calibrated with aqueous calibration standards.

6. said volatile chemical monitoring system of claim 1 calibrated with gas permeation tube.

7. said volatile chemical monitoring system of claim 1 wherein the sample chamber is located under the static water level of the well and water is introduced into the sample chamber using hydrostatic pressure.

8. said volatile chemical monitoring system of claim 1 calibrated with gaseous standards.

9. Volatile chemical monitoring system comprising:
(a) an analytical chamber containing a sensor,
(b) a valve to divert gas or air into an evacuated cylinder,
(c) an evacuated cylinder for collecting gas or air samples,
(d) a means of delivering volatile compounds in a stream of a gas or air to the volatile chemical monitoring system,

10. Said volatile chemical monitoring system of claim 9 comprising of an analytical chamber with a non-destructive sensor.

11. said volatile chemical monitoring system of claim 9 wherein a diaphragm or centrifugal pump is the means of delivering the volatile compounds in a stream of air.
